(19) United States
(12) Patent Application Publication
(10) Pub. No.: US 2010/0305411 A1
(43) Pub. Date: Dec. 2, 2010

(54) Control of operating characteristics of devices relevant to distance of visual fixation using input from respiratory system and/or from eyelid function

(75) Inventor: Scanlan Paul, London (GB)

Correspondence Address: GUNN, LEE & CAVE, P.C., 300 CONVENT ST., SUITE 1080, SAN ANTONIO, TX, 78205, US

(73) Assignee: VP UK IP LIMITED, Birmingham (GB)

(21) Appl. No.: 12/377,431

(22) PCT Filed: Aug. 14, 2007
(86) PCT No.: PCT/GB07/03068

§ 371 Date: Aug. 18, 2010

(30) Foreign Application Priority Data
Aug. 15, 2006 (GB) 0616189.7

(51) Int. Cl. A61B 5/00

(52) U.S. Cl. 600/301

(57) ABSTRACT

Determination of distance of visual fixation using input from respiratory system and/or from eyelid function, for the purpose of controlling applications including the focus of image capture and viewing devices.

The invention relates to an apparatus and method for using respiratory and/or eyelid function data as a control system for applications which would benefit from a determination of a person's distance of visual fixation, such as to control the focusing of image capture and viewing devices.

### BACKGROUND OF THE INVENTION

Applicant claims priority under 35 U.S.C. §119 of International Patent Application No. PCT/GB2007/003068, filed Aug. 14, 2007, which claims priority to United Kingdom (GB) Patent Application No. 0616189.7, filed Aug. 15, 2006.

### TECHNICAL FIELD

The invention relates to an apparatus and method for using respiratory and/or eyelid function data as a control system for applications which would benefit from a determination of a person's distance of visual fixation, such as to control the focusing of image capture and viewing devices.

### BACKGROUND ART

Image capture devices include still and video cameras. Auto-focus systems which adjust variable lens arrangements are a well known feature of some image capture devices, designed to obtain and maintain correct focus on a subject without the user's manual intervention.

In many situations, manual focusing results in significantly sharper focusing than with an auto-focus system. More specifically, due to the discrete nature of auto-focus sensors and attendant focusing offsets and errors, there is a resulting loss of resolution using digital auto-focus compared to analogue manual focusing by eye. Low light levels and low contrast subjects present difficulties for auto-focus systems. Similarly, errors may be introduced by high contrast bars aligning with the axis of the sensors and by the need to guess the real focusing point between sensors. Sensor size, speed, noise and battery issues also introduce limitations. Active auto-focus sensing systems use infrared and similar distance measuring technology to improve the accuracy of auto-focus sensors in difficult conditions. However, due to power and distance measuring accuracy limitations, active infrared does not work well at long distances. In addition, if there is a window between the image capture device and the subject, this may present difficulties for the distance measuring technology used in active auto-focus sensing systems.

Viewing devices with variable lens arrangements include binoculars, telescopes, microscopes, night vision goggles and spectacles such as the recently invented liquid crystal bifocals. Liquid crystal bifocals vary from near to far focus by application of a varied electric current to the liquid crystal. These bifocals require an input to determine whether it is appropriate to provide near or far focus in the same way that binoculars, telescopes etc also require such input. To the extent that this input is manual, this is inconvenient for the user. To the extent that this is an auto-focus input, using electronics and optics to make an assessment of whether the user's attention is directed at a near or distant object, the same limitations apply as for current auto-focus devices for still and video cameras, as described in the paragraph above.

### SUMMARY OF THE INVENTION

As discovered by the inventor, the respiratory system directly influences the visual system. The inventor discovered that pressure from the respiratory system presses on the rear of the eyeball, changing the eyeball's length from front to back, thereby altering the focus of the eye. Increased pressure from the respiratory system pushing on the back of the eyeball reduces the length of the eyeball for better distance vision. A decrease in this pressure increases the length of the eyeball for better close-up vision. Thus when a person changes from viewing an object in the distance to instead viewing an object close-up, there is a corresponding change in pressure in the respiratory system. Changes in pressure in the respiratory system are achieved by changes to variables including the depth and timing of in-breath and out-breath. The inventor also discovered that the depth of a person's in-breath is affected by whether the person's eyes are wide open or eyelid squinting. When a person opens his or her eyes widely, this prompts a deeper in-breath. When a person eyelid squints, this prompts a more shallow in-breath. This effect of eyelid function on the respiratory system can influence the pressure within the respiratory system which has a corresponding influence on the length of the eyeball from front to back, as described above. A person will generally have his or her eyes open more widely when looking at objects in the distance compared to when looking at closer objects. A disadvantage of existing image capture and viewing devices, and in particular the auto-focus systems used as part of those devices, is that they do not take into account the influence of respiratory and eyelid function on the user's visual system.

An object of this invention is to communicate to an auto-focus system (for image capture and viewing purposes) the user's attention shifts from near to far. Accordingly, this invention communicates commands to the auto-focus system based on real-time respiratory and/or eyelid function data which varies according to whether the photographer's, video camera operator's, spectacle wearer's etc attention is on a near or distant object.

By tracing the user's shift in attention from near to far objects as the shift happens and communicating this to an auto-focus system, the invention will make the process of auto-focus faster, more accurate and more reliable, particularly in difficult conditions such as where there are low light levels. The invention generates an input which alone or in concert with other inputs can control an auto-focus system. The invention communicates to an image capture or viewing device information concerning the influence of the user's respiratory system and eyelid function on the user's visual system, thereby assisting in the task of focusing the image capture or viewing device.

### BRIEF DESCRIPTION OF THE DRAWINGS

An example of the invention will now be described by reference to the accompanying diagrams:

FIG. 1 shows a schematic diagram of the major components of a preferred embodiment of the invention;

FIG. 2 shows the context in which a preferred embodiment of the invention may be used;

FIG. 3 shows the inverse relationship between pressure in the respiratory system pushing on the rear of the eyeball and distance of visual fixation;

FIG. 4 shows a diagram of a user's breathing wave form showing an increase and decrease of in-breath relative to the changing distance of the distance of visual fixation (subject of attention) and the corresponding focus command to adjust the image capture or viewing device; and

FIG. 5 shows a diagram of a user's eyelid function relative to the changing distance of the distance of visual fixation and the corresponding focus command to adjust the image capture or viewing device.

### DETAILED DESCRIPTION OF THE INVENTION

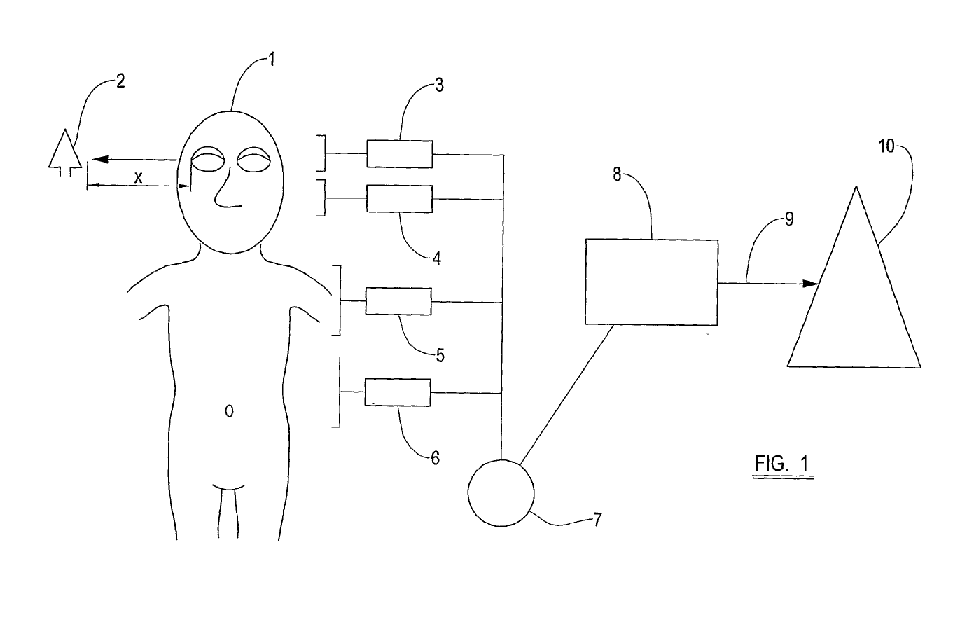

FIG. 1 shows a schematic diagram of the major components of a preferred embodiment of the invention. More specifically, FIG. 1 shows a user 1 viewing an object 2 at a given distance from the user. Input sensors collect data from the eyelids 3, nose 4, chest 5 and abdomen 6 of the user. A signal unit 7 collects the respiratory and eyelid function data and communicates this data to the computation unit 8. At the computation unit, calculations are performed on this data to determine the user's distance of visual fixation. Based on the user's distance of visual fixation, the computation unit sends a command 9 to control an operating characteristic of a device 10, such as the focussing mechanism of a camera.

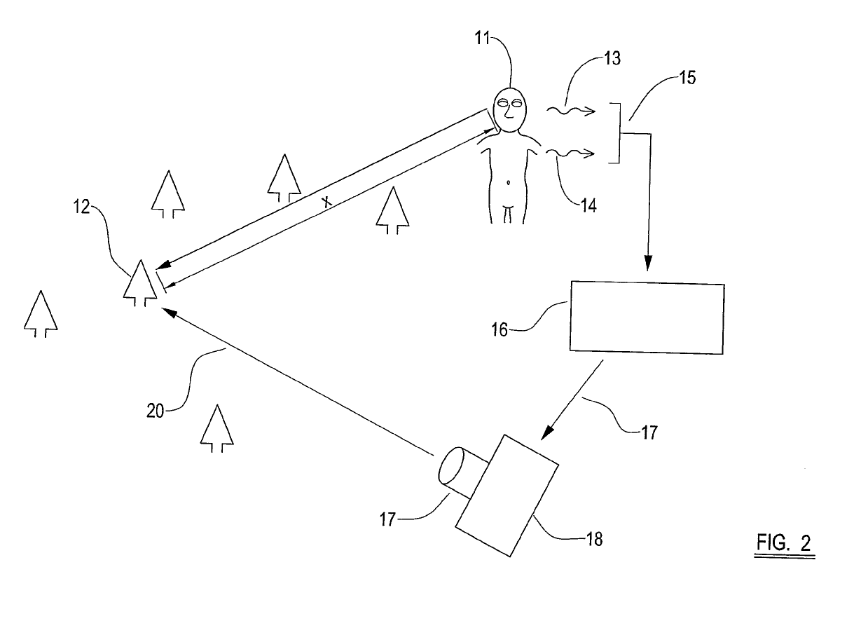

FIG. 2 shows a context in which a preferred embodiment of the invention may be used. More specifically, FIG. 2 shows a user 11 viewing an object 12 at a given distance, x metres from the user. The user's eyelid function data 13 and respiratory function data 14 are gathered by sensors 15 and communicated to a computation unit 16. The computation unit compares the user's respiratory function data and eyelid function data to stored data and calculates the user's distance of visual fixation. The computation unit sends a command 17 to a device, in this case a camera 18, controlling an operating characteristic of that device, in this case the focussing mechanism of the camera 19, which accurately focuses on the object 20 as a result of the invention's calculation of the user's distance of visual fixation.

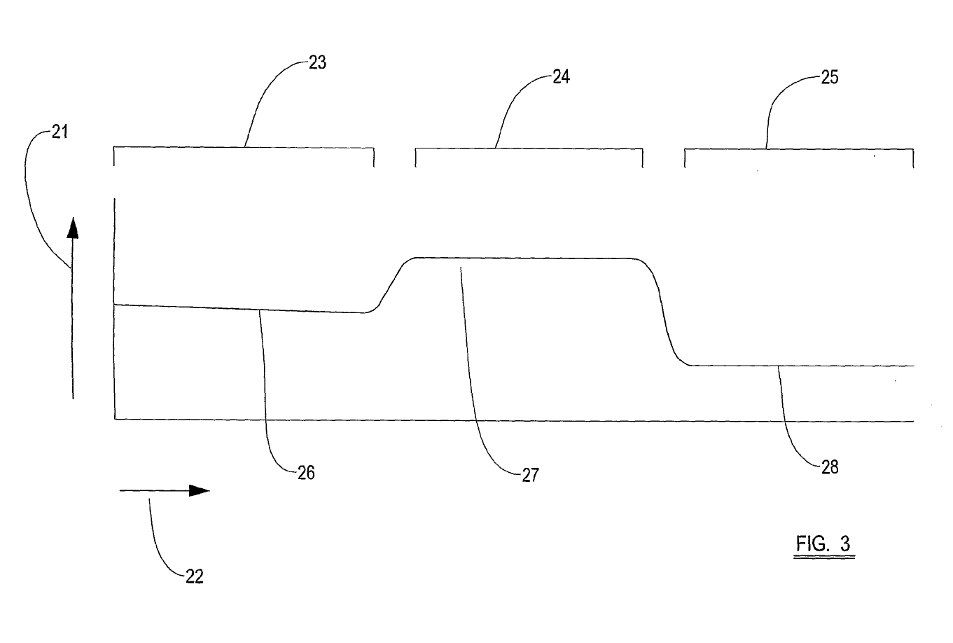

FIG. 3 shows the inverse relationship between pressure in the respiratory system pushing on the rear of the eyeball and distance of visual fixation. More specifically, by way of example, FIG. 3 shows a graph of pressure in the respiratory system 21 pushing against the rear of the eyeball as that pressure changes over time 22. Over the time period shown in the graph in FIG. 3, the person changes from viewing an object in the middle distance 23 to instead viewing an object in the far distance 24 and then changes to viewing an object that is close up 25. FIG. 3 shows that the pressure increases as a person changes from viewing an object in the middle distance 26 to instead viewing an object in the far distance 27 and that the pressure decreases as the person changes to viewing an object that is close up 28.

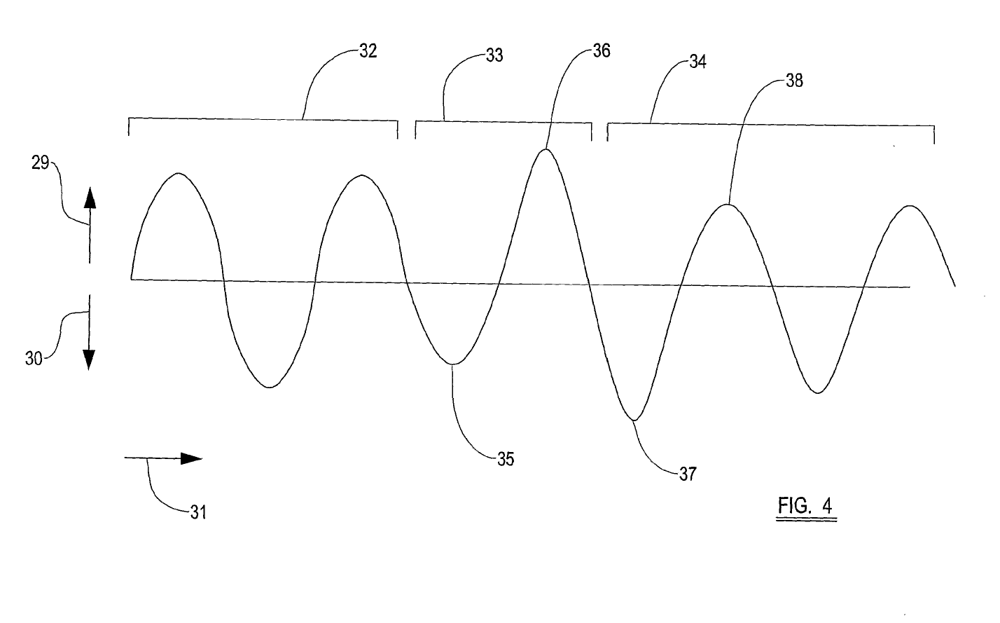

FIG. 4 shows a diagram of a user's breathing wave form showing an increase and decrease of in-breath relative to the changing distance of the distance of visual fixation and the corresponding focus command to adjust the image capture or viewing device. More specifically, by way of example, FIG. 4 shows a graph of depth of in-breath 29 and out-breath 30 and how that changes over time 31. Over the time period shown in the graph in FIG. 4, the person changes from viewing an object in the middle distance 32 to instead viewing an object in the far distance 33 and then changes to viewing an object that is close up 34. As shown in FIG. 4, when a person changes from viewing an object in the middle distance to instead viewing an object in the far distance, this is manifested in the user's breathing wave form by a decreased out-breath 35 and increased in-breath 36. The corresponding focus command to adjust the image capture or viewing device for both a decreased out-breath 35 and an increased in-breath 36 is to adjust the focus for an increased distance to the subject. As shown in FIG. 4, when a person changes from viewing an object in the far distance 33 to instead viewing an object that is close up 34, this is manifested in the user's breathing wave form by an increased out-breath 37 and decreased in-breath 38. The corresponding focus command to adjust the image capture or viewing device for both an increased out-breath 37 and a decreased in-breath 38 is to adjust the focus for a decreased distance to the subject.

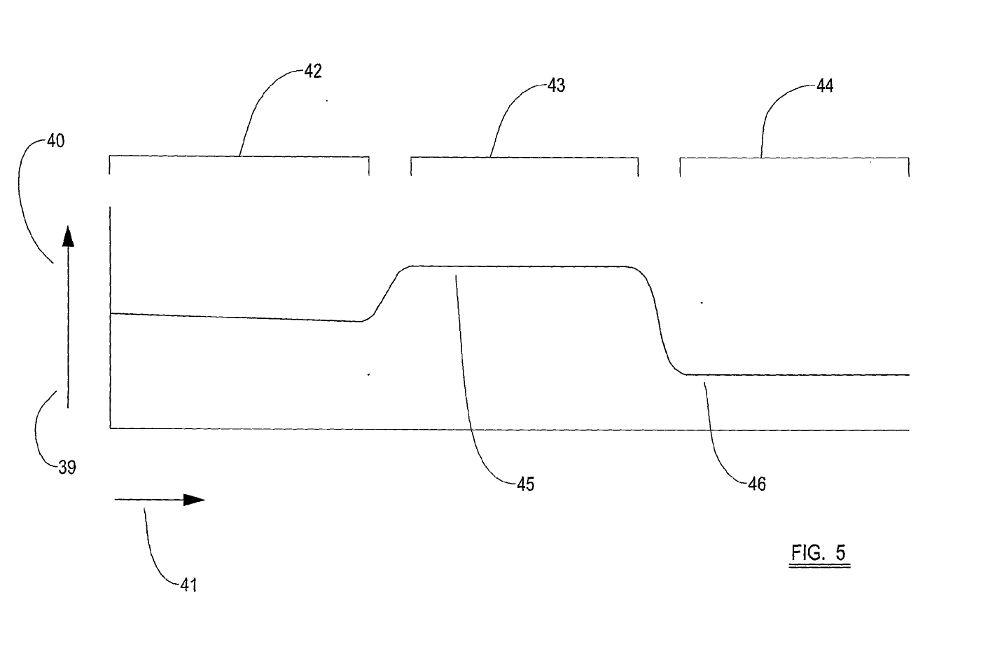

FIG. 5 shows a diagram of a user's eyelid function relative to the changing distance of the distance of visual fixation and the corresponding focus command to adjust the image capture or viewing device. More specifically, by way of example, FIG. 5 shows a graph of how wide the eye is open (from eyelid squinting 39 to fully open 40) and how that changes over time 41. Over the time period shown in the graph in FIG. 5, the person changes from viewing an object in the middle distance 42 to instead viewing an object in the far distance 43 and then changes to viewing an object that is close up 44. As shown in FIG. 5, when a person changes from viewing an object in the middle distance 42 to instead viewing an object in the far distance 43, this is manifested in the user's eyelid function by an increase 45 in how wide the eye is open. The corresponding focus command to adjust the image capture or viewing device for an increase in how wide the eye is open 45 is to adjust the focus for an increased distance to the subject. As shown in FIG. 5, when a person changes from viewing an object in the far distance 43 to instead viewing an object that is close up 44, this is manifested in the user's eyelid function by a decrease in how wide the eye is open 46. The corresponding focus command to adjust the image capture or viewing device for a decrease in how wide the eye is open 46 is to adjust the focus for a decreased distance to the subject.

### A Preferred Embodiment

Preferably the apparatus consists of one or more input sensors, one or more signal units, a computation unit and a variable lens arrangement. The variable lens arrangement is part of an image capture or viewing device (and includes, for example, a standard auto-focus 35 mm lens as well as the adjustable lens of liquid crystal bifocals).

The purpose of the input sensors is to detect the user's real-time respiratory and eyelid function data. Preferably the input sensors are placed around the abdomen, chest, nose and/or eyelids of the user. The sensors around the abdomen and chest detect the magnitude and timing of expansion and contraction of these areas for the purpose of detecting respiratory function. The nasal sensor detects the magnitude and timing of air flow into and out of the nose preferably by sound detection, such as through the use of a piezoelectric device. The eyelid sensor detects the timing of blinking and the degree to which the eye is fully open or eyelid squinting at any given time. Such sensors of physiological data are well known to those skilled in the art of biofeedback and biomonitoring.

For the avoidance of doubt, blinking refers to the contraction of the fast twitch fibres in the palpebral portion of the orbicularis oculi muscle, whilst eyelid squint refers to the contraction of the orbital portion of that muscle (though the action of eyelid squinting may to some lesser extent also engage the palpebral portion). An appropriate eyelid sensor to measure the degree of eyelid squinting may take the form of an electromyography apparatus attaching surface electrodes to the skin close to the eyelids to measure electromyography potentials (such as described in Sheedy J E, Gowrisankaran, S and Hayes J R, Blink rate decreases with eyelid eyelid squint, Optom Vis Sci 2005; Vol 82. No. 10; 905-911). Eyelid squint, which commonly can be referred to as narrowing the eyes, is apparent as a change in the vertical dimension of the palpebral fissure (also known as ocular aperture). Therefore another appropriate eyelid sensor may take the form of a video based assessment of changing palpebral fissure height, which serves to detect both eyelid squinting and blinking, using apparatus such as the ISCAN eye tracker produced by ISCAN Incorporated, Burlington, Mass., USA.

One or more signal units collect the respiratory and eyelid function data and communicate this data in real time to the computation unit. This communication can be by wires or wireless means such as using infrared technology.

The computation unit receives the physiological data from the signal unit. The computation unit compares the incoming physiological data to stored data. The computation unit determines whether the user's respiratory and eyelid function is changing and if so the magnitude, direction and rate of that change. Changes detected in the user's respiratory and eyelid function are communicated by the computation unit to the variable lens arrangement in the form of a command affecting the focus of the variable lens arrangement.

Increased depth of the user's in-breath is communicated from the computation unit to the variable lens arrangement as a command to adjust the focus for an increased distance to the subject. Decreased depth of in-breath is communicated to the variable lens arrangement as a command to adjust the focus for a decreased distance to the subject. Rapid exhalation of air through the user's nose is communicated to the variable lens arrangement as a command to adjust the focus for a decreased distance to the subject. Increased eyelid squint is communicated to the variable lens arrangement as a command to adjust the focus for a decreased distance to the subject. Increased opening of eyelids is communicated to the variable lens arrangement as a command to adjust the focus for an increased distance to the subject. In this way, the invention causes the user's respiratory and/or eyelid function to influence an image capture or viewing device through changing the variable lens arrangement of an image capture or viewing device.

### An Alternative Embodiment

An alternative embodiment includes one or more input sensors to detect the user's state of accommodation (such as an infrared optometer as described in U.S. Pat. No. 4,162,828 and U.S. Pat. No. 4,533,221) which, combined with respiratory and/or eyelid function data, is used to provide biofeedback for accommodation training Accommodation is the ability of the eye to adjust to focus on objects at various distances. Biofeedback describes the process of monitoring and communicating information about physiological processes, such as respiration and blood circulation, to enable the patient to be contemporaneously aware of changes in those physiological processes and also to assist with voluntary self regulation (or training) of those processes. The goal of biofeedback is to enable the patient to improve beyond normal function towards an optimal level, or, where there is impaired functioning, to reduce or eliminate the symptoms of impairment. Accordingly, this embodiment of the invention communicates to the patient his or her respiratory system and eyelid function data at the same time as communicating to the patient his or her state of accommodation.

Prior attempts have been made to reduce or cure impairments of the visual system such as myopia. At least one current biofeedback device called the Accommotrac® (based on U.S. Pat. No. 4,533,221) seeks to provide awareness to the patient of his or her state of accommodation. Accommotrac is premised on the basis that it seeks to assist the patient with voluntary self regulation of a muscle within the eye called the ciliary muscle. Accommotrac provides an audio signal which varies according to the patient's state of accommodation but does not provide other information about the patient's physiological processes. No existing biofeedback device which provides awareness to the patient of his or her state of accommodation also makes the patient aware of changes in respiratory and/or eyelid function.

Prior attempts using biofeedback devices to reduce or cure impairments of the visual system have not been totally satisfactory because they have not taken into account the direct influence of the respiratory system on the visual system. As discovered by the inventor, the visual system is directly influenced by changes in pressure within the respiratory system. Pressure changes within the respiratory system alter the length of the eyeball front to back, which alters the focusing characteristics of the eye. Prior to the inventor's discovery, it was not known that lower than normal pressure within the respiratory system is the main cause of myopia and that higher than normal pressure within the respiratory system is the main cause of hyperopia.

This alternative embodiment of the current invention can provide biofeedback allowing a patient to be contemporaneously aware of respiratory system variables and eyelid function relative to the patient's state of accommodation. For these purposes, the term accommodation is used to describe not only the effect of the ciliary muscle on the lens of the eye but also the effect of the respiratory system on the length of the eyeball. Prior to the inventor's discovery, only the effect of the ciliary muscle on the lens was thought to be important in causing accommodation. This embodiment of the current invention provides biofeedback which allows the patient to improve beyond normal visual function towards an optimal level, or, where there is impaired functioning such as myopia or hyperopia, to reduce or eliminate these symptoms of impairment through voluntary self regulation (or training) of respiratory system variables and eyelid function relevant to accommodation.

By making a patient aware not only of his or her state of accommodation but also giving biofeedback about the patient's respiratory and/or eyelid function, this embodiment of the current invention will make the process of voluntary self regulation (or training) of visual function faster and more reliable. Where the patient seeks to improve beyond normal visual function towards an optimal level, or, where there is impaired functioning such as myopia or hyperopia, to reduce or eliminate these symptoms of impairment, the invention will speed up the process by making apparent to the patient an important (but previously ignored) determinant of clear vision, that being the patient's respiratory and/or eyelid function.

Preferably, this alternative embodiment of the invention consists of one or more input sensors to detect the user's real-time respiratory and/or eyelid function data, one or more input sensors to detect the user's state of accommodation, one or more signal units, a computation unit and two or more output units. The input sensors to detect the user's real-time respiratory and/or eyelid function data are as described above for the preferred embodiment of the invention. Preferably the input sensor to detect the user's state of accommodation is an infrared optometer such as that described in U.S. Pat. No. 4,162,828 and U.S. Pat. No. 4,533,221.

One or more signal units collect the data from the sensors and communicate this in real time to the computation unit. This communication can be by wires or wireless means. The computation unit receives the respiratory and/or eyelid function data from the signal units. The computation unit compares the incoming physiological data to stored data. The computation unit determines whether the user's respiratory and/or eyelid function is changing and if so the magnitude, direction and rate of that change. Changes detected in the user's respiratory and/or eyelid function are communicated by the computation unit to one or more output units. The output units indicate the changes to the patient either in the form of a changing tone, changing tactile display or some other means that can be sensed by the patient.

The computation unit also receives the state of accommodation data from the signal units. The computation unit compares the incoming accommodation data to stored data. The computation unit determines whether the user's state of accommodation is changing and if so the magnitude, direction and rate of that change. Changes detected in the user's state of accommodation are communicated by the computation unit to one or more output units. The output units indicate the changes to the patient either in the form of a changing tone, changing tactile display or some other means that can be sensed by the patient. The detection and communication of the user's state of accommodation can be achieved using the methods and apparatus described in U.S. Pat. No. 4,533,221.

When using this embodiment of the current invention, the patient is made aware of both his or her state of accommodation and his or her respiratory and/or eyelid function. The latter are a major determinant of the former. Therefore, when using this embodiment of the current invention, the patient can, through voluntary self regulation (or training) of the respiratory and/or eyelid function processes, learn to control his or her state of accommodation.

An alternative embodiment includes a visual acuity array, such as that described in U.S. Pat. No. 4,533,221. The visual acuity array can be used as a simple means of detecting the user's state of accommodation for comparison to biofeedback from the user's respiratory and/or eyelid function.

### Another Alternative Embodiment

An alternative embodiment includes the use of respiratory and/or eyelid function data to control interactive visual displays. Interactive visual displays include three-dimensional video games where the perspective shown on-screen changes according to input from the player. For example, using a keystroke or manipulation of a joystick, a player can input a direction to turn to the left or to the right, which prompts the on-screen display to show a different view from the initial position. Prior attempts at interactive visual displays have included a zoom function where the viewer of the display can manually input a zoom in or zoom out command so as to change the perspective shown on screen from a distant view to a more close-up view and vice versa. Prior attempts to simulate real three dimensional perspectives have not been totally satisfactory because they have not taken into account the direct influence of the respiratory system on the visual system but have instead relied on either a fixed perspective or manual input of a zoom in or zoom out command.

Other interactive visual displays include interactive displays appearing on computer screens, television screens, video screens and in movie theatres or other projections.

As discovered by the inventor, the visual system is directly influenced by changes in pressure within the respiratory system. Pressure changes within the respiratory system alter the refractive state of the eye. When a person's attention is drawn from a near to a distant object, this prompts an in-breath and corresponding increase in pressure in the respiratory system, shortening the front-to-back length of the eyeball for optimal distance vision. There is a corresponding decrease in pressure in the respiratory system (generally achieved by a release of air through the nose) when a person's attention is drawn from a distant object to a near object.

This alternative embodiment of the current invention allows for control of an interactive visual display by input from the viewer's respiratory system. This alternative embodiment of the current invention transforms input from the viewer's respiratory system into commands which manipulate the on-screen perspective, such as a zoom in or zoom out command.

Preferably, this alternative embodiment of the invention consists of one or more input sensors, one or more signal units, a computation unit and an output to an interactive visual display. The input sensors and signal units are for respiratory and/or eyelid function data as described above in relation to a preferred embodiment. A computation unit, as described above in relation to a preferred embodiment, receives physiological data from the signal unit as the user completes the interactive task such as playing a role-playing computer game. The computation unit compares the incoming physiological data to stored data. The computation unit determines whether the user's respiratory and/or eyelid function is changing and if so the magnitude, direction and rate of that change. Changes detected in the user's respiratory and/or eyelid function are communicated by the computation unit as an output command affecting the interactive visual display. The interactive visual display zooms in, zooms out or remains with the current field of view depending on the output command received from the computation unit. Increased depth of in-breath is communicated to the interactive visual display as a command to zoom out. Decreased depth of in-breath is communicated to the interactive visual display as a command to zoom in. Rapid exhale of air through the user's nose is communicated to the interactive visual display as a command to zoom in. Increased eyelid squint is communicated to the interactive visual display as a command to zoom in. Increased opening of eyelids is communicated to the interactive visual display as a command to zoom out. In this way, this alternative embodiment of the invention causes the respiratory and/or eyelid function to influence image display through communicating commands to an interactive visual display.

### A Further Alternative Embodiment

An alternative embodiment includes the use of respiratory and/or eyelid function data to control a device which modifies the pressure in the respiratory system. As discovered by the inventor, myopia is a condition which occurs when there is lower than normal pressure pushing on the rear of the eyeball and hyperopia is a condition which occurs when there is higher than normal pressure pushing on the rear of the eyeball. To correct these refractive errors, the pressure in the respiratory system can be modified by a device which is, for example, held in the mouth, in the same fashion as a regulator used by scuba divers, and either pumps air into or out of the respiratory system. By using respiratory and/or eyelid function data to determine whether the user's distance of visual fixation is on a distant or near object, a command can be given to the device which modifies the pressure in the respiratory system to either increase the pressure (to assist a myopic user to see distant objects more clearly) or decrease the pressure (to assist a hyperopic user to see close-up objects more clearly).

### Additional Alternative Embodiments

An alternative embodiment includes a sensor directly measuring pressure within the respiratory system such as a pressure sensor held in the mouth, held between the lips or implanted into a paranasal sinus chamber.

An alternative embodiment includes a sensor which measures pressure within the respiratory system by detecting changes in the sound of the user's breathing or output of the vocal system. For example, as discovered by the inventor, the sound of a person's humming changes (reflecting a change in pressure in the respiratory system) as the person changes their distance of visual fixation. A person's humming sounds different depending upon whether the person is looking at a near or distant object. This change in sound can be used to determine distance of visual fixation and applied to control the operating characteristics of relevant devices such as image capturing devices.

An alternative embodiment includes a sensor that monitors heart rate in order to compare this input to the changing respiratory and/or eyelid function parameters. As the user's heart rate increases, such as with exercise, an increasingly deep in-breath is anticipated irrespective of point of visual fixation and therefore the detection of an increased heart rate would dampen the command associated with an increasing in-breath.

An alternative embodiment includes a motion sensor such as used in pedometers in order to compare this input to the changing respiratory and/or eyelid function parameters. Increased motion will generally relate to an increased heart rate and as the heart rate increases an increasingly deep in-breath is anticipated irrespective of point of visual fixation. Therefore, the detection of increased motion would dampen the command associated with an increasing in-breath.

An alternative embodiment uses an electroencephalogram sensor or nerve sensor to detect electrical activity of the brain or nervous system controlling respiratory and/or eyelid functions. These electrical impulses can be used as input data instead of or in addition to data collected by other sensors.

An alternative embodiment includes a plus lens in between the eye of the user and the subject being viewed. The use of a plus lens exaggerates the sensitivity of the user's respiratory and eyelid function response to shifts in attention from near to more distant objects. For example, if a plus lens of strength +1.0 is used, the user's normal range of focus from close up to the eye to an optically infinite distance (and corresponding respiratory and eyelid function) is condensed by use of the lens to a distance from close up to the eye to one metre from the eye. As a result, when using a +1.0 lens, the user's shift in attention from an object close to the eye to an object one metre from the eye has a corresponding respiratory and eyelid function signature/response equivalent to a shift in attention from an object close to the eye to an object in the far distance (e.g. 20 metres away) under conditions without the plus lens.

An alternative embodiment includes a minus lens in between the eye of the user and the subject being viewed. The use of a minus lens diminishes the sensitivity of the user's respiratory and eyelid function response to shifts in attention from near to more distant objects. For example, if a minus lens of strength −1.0 is used, only that portion of the user's range of focus from close up to the eye to one metre away (and the corresponding limited range of respiratory and eyelid function) is used when viewing objects from close up to the eye to an optically infinite distance from the eye. As a result, when using a −1.0 lens, the user's shift in attention from an object close to the eye to an object 20 metres from the eye has a corresponding respiratory and eyelid function signature/response equivalent to a shift in attention from an object close to the eye to an object one metre away under conditions without the minus lens.

An alternative embodiment includes a computation unit that can be calibrated for individual users. Individual users have different respiratory system parameters due to factors such as lung size and aerobic fitness levels. Similarly, individual users may have different eyelid function parameters due to genetic differences dictating the shape of the bone, muscle and other tissue arrangement around the eye (such as the shape of the orbicularis oculi). These differences between individual users can be taken into account by the computation unit for the purpose of determining the appropriate commands to communicate to the variable lens arrangement. Also, different users may have different respiratory and/or eyelid function responses due to conditions such as myopia, hyperopia or presbyopia. To enable these differences between users to be taken into account, calibration can be achieved by the computation unit taking input from a user looking at certain objects at known distances.

An alternative embodiment includes a light sensor and any other sensors used in auto-focus devices.

An alternative embodiment logs the user's respiratory and/or eyelid function data over time. This enables analysis of a user's pattern of near and distant viewing which may be correlated against, for example, the user's work or sport activities or changes in the user's health including visual health. Confined visual environments have been shown in animal studies to induce myopia. Similarly, prolonged service on submarines has been correlated in humans with increased degrees of myopia. For journeys in space lasting several years, logging the astronaut's respiratory and/or eyelid function data in order to prescribe appropriate visual exercises, such as increased periods looking into the distance, may help prevent deterioration of visual function. Excessive close up work, such as extended periods of computer use at close range, is associated with the onset of myopia. Eyelid squinting is associated with an increased incidence of myopia. Eyelid squinting is also associated with a breathing pattern characterised by shallower in-breaths. By logging a user's respiratory and/or eyelid function data over time, and hence generating a record of distance of visual fixation over time, the user's level of exposure to risk factors associated with myopia can be monitored and, where appropriate, patterns of behaviour can be modified accordingly.

An alternative embodiment uses respiratory and/or eyelid function data to control range-dependant devices such as weapons. For example, if a user is aiming a weapon, rather than manually inputting the required distance between the user and the target, the appropriate range can be determined by using respiratory and/or eyelid function data to calculate the user's distance of visual fixation when looking at the target.

An alternative embodiment uses respiratory and/or eyelid function data to control distance calculations for surveying purposes. For example, if a user is surveying a site, rather than manually measuring each required distance between the user and specific points or objects, the distance can be determined by using respiratory and/or eyelid function data to determine the user's distance of visual fixation when looking at each specific point or object.

An alternative embodiment uses respiratory and/or eyelid function data to control distance calculations for controlling vehicles including those used in road, rail, air and sea transport. For example, if an aircraft pilot is watching a designated specific point or set of points on a runway as the aircraft approaches the runway, the pilot's respiratory and/or eyelid function data can be used to determine the aircraft's distance from each specific point and accordingly, based on those distance calculations, control the operating characteristics of the aircraft. Similarly, if a driver wishes to park in a space between two other vehicles, the driver can look at a designated specific point or set of points on each of the two other vehicles and the driver's respiratory and/or eyelid function data can be used to determine the car's distance from each of the two vehicles and accordingly, based on those distance calculations, control the operating characteristics of the car to enable the parking manoeuvre to be successfully carried out.

An alternative embodiment uses respiratory and/or eyelid function data to provide additional safety in vehicles, including those used in road, rail, air and sea transport. Respiratory and/or eyelid function, as indicators of distance of visual fixation, can be compared to the speed of the vehicle. If the respiratory and/or eyelid function indicate that the user's (driver's, pilot's, captain's etc) attention is, for any length of time, not at an appropriate distance given the speed at which the vehicle is travelling, this can trigger one or more events. These events can include a warning signal (being communicated to the user and/or to a remote person or machine) and/or an automatic reduction of the speed of the vehicle either to a stop or to a speed appropriate to the user's distance of visual fixation as indicated by the user's respiratory and/or eyelid function.

An alternative embodiment uses respiratory and/or eyelid function data to control option selection when the user is presented with options at varying distances from the user. Rather than manually inputting the chosen option, the user's choice can be determined by using respiratory and/or eyelid function data to calculate the user's distance of visual fixation when looking at the chosen option.

An alternative embodiment uses respiratory and/or eyelid function data to give instructions to devices such as robotic lawn mowers. Rather than manually inputting the area of lawn to be mowed, the user's choice can be communicated to the robot by using respiratory and/or eyelid function data to calculate the user's distance of visual fixation when the user is looking at areas that the user wishes to have mown.

An alternative embodiment includes the use of respiratory and/or eyelid function data for the purpose of avoiding the onset of, delaying the onset of, stabilising or reversing myopia.

An alternative embodiment includes the use of respiratory and/or eyelid function data to control interactive visual displays for the purpose of avoiding, delaying, stabilising or reversing myopia. As noted above, excessive close up work, such as extended periods of computer use at close range, is associated with the onset of myopia. Eyelid squinting is associated with an increased incidence of myopia. Eyelid squinting is also associated with a breathing pattern characterised by shallower in-breaths. When viewing an interactive visual display, the user's respiratory and/or eyelid function data can be monitored such that a decrease in distance of visual fixation and/or increase in eyelid squint prompts a warning to be displayed on the interactive visual display. In addition or as an alternative to the display of a warning, a zoom setting on the interactive visual display can be controlled by input from the user's respiratory and/or eyelid function data such that the font size of text or image size is increased so as to prompt a decrease in or elimination of the user's eyelid squint and/or an increase in the user's distance of visual fixation.

To ensure best results when using the invention, users are advised to breathe through their nose, keeping their mouths closed. Users are also recommended to retain a stable posture, preferably an upright rather than slouched posture.

When used in this specification and claims, the terms “comprises” and “comprising” and variations thereof mean that the specified features, steps or integers are included. The terms are not to be interpreted to exclude the presence of other features, steps or components.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilised for realising the invention in diverse forms thereof

1-43. (canceled)

44. A method of controlling an operating characteristic of a device wherein that operating characteristic is related to a person's distance of visual fixation, characterised in that the method includes detecting selected physiological data of the person, which data is representative of changes in the distance of visual fixation of the person controlling the operating characteristic in response to the physiological data of the person.

45. A method according to claim 44 wherein the physiological data includes at least one of respiratory function data and eyelid function data.

46. A method according to claim 44 where the determination of changes in distance of visual fixation is used to control the focus of image viewing and image capture devices.

47. A method according to claim 44 where the determination of changes in distance of visual fixation is used to control the focus of image capture for a camera.

48. A method according to claim 44 where the determination of changes in distance of visual fixation is used to control the focus of at least one of:
spectacles such as liquid crystal bifocals;
binoculars;
telescopes;
microscopes;
night vision goggles.

49. An apparatus for controlling an operating characteristic of a device wherein that operating characteristic is relevant to a person's distance of visual fixation, characterised in that the apparatus includes a computation unit which is operable to control the operating characteristic in response to an input from a sensor which collects physiological data of the person, to determine changes in the distance of visual fixation of the person.

50. An apparatus according to claim 49 wherein the physiological data includes at least one of the person's respiratory function data and eyelid function data.

51. An apparatus according to claim 49 which includes a sensor directly measuring pressure within the respiratory system such as a pressure sensor held in the mouth, held between the lips or implanted into a paranasal sinus chamber.

52. An apparatus according to claim 49 which includes at least one of:
a sensor which measures pressure within the respiratory system by detecting changes in the sound of the user's breathing or output of the vocal system;
a sensor that monitors heart rate in order to compare this input to the changing respiratory and/or eyelid function parameters;
a motion sensor such as used in pedometers in order to compare this input to the changing respiratory and/or eyelid function parameters; and
an electroencephalogram sensor or nerve sensor to detect electrical activity of the brain or nervous system controlling respiratory and/or eyelid functions.

53. An apparatus according to claim 49 which includes at least one plus lens in between the eye of the user and the subject being viewed to exaggerate the sensitivity of the user's respiratory and eyelid function response to shifts in attention from near to more distant objects.

54. An apparatus according to claim 49 which includes one or more minus lenses in between the eye of the user and the subject being viewed to diminish the sensitivity of the user's respiratory and eyelid function response to shifts in attention from near to more distant objects.

55. An apparatus according to claim 49 wherein the computation unit can be calibrated for individual users.

56. An apparatus according to claim 49 which includes a light sensor and any other sensors used in auto-focus devices.

57. An apparatus according to claim 49 which logs the user's respiratory and/or eyelid function data over time.

58. An apparatus according to claim 49 which includes one or more input sensors or visual acuity arrays to detect the user's state of accommodation such as an infrared optometer.

59. An apparatus according to claim 49 for use in biofeedback accommodation training

60. An apparatus according to claim 49 to control at least one of interactive visual displays including video games and other displays appearing on computer screens, television screens, video screens and in movie theatres or other projections;
a device which modifies the pressure in the respiratory system;
a device which modifies the pressure in the respiratory system for the purpose of correcting refractive errors;
range-dependant devices such as weapons;
distance calculations for surveying purposes; and
option selection when the user is presented with options at varying distance calculations for the purpose of controlling vehicles including those used in road, rail, air and sea transport distances from the user.

61. An apparatus according to claim 49 to control interactive visual displays for the purpose of avoiding, delaying, stabilising or reversing myopia by warning of a decrease in distance of visual fixation.

62. An apparatus according to claim 49 to control interactive visual displays for the purpose of avoiding, delaying, stabilising or reversing myopia by warning of an increase in eyelid squint.

63. An apparatus according to claim 49 to control interactive visual displays for the purpose of avoiding, delaying, stabilising or reversing myopia by increasing the font size of text or image size in response to a decrease in distance of visual fixation or an increase in eyelid squint.
